(19) United States
(12) Patent Application Publication
(10) Pub. No.: US 2024/0082052 A1
(43) Pub. Date: Mar. 14, 2024

(54) Targeted Temperature Management Systems, Pads, and Methods Thereof

(71) Applicant: C. R. Bard, Inc., Franklin Lakes, NJ (US)

(72) Inventors: Younggeun Cho, Covington, GA (US); Cecille A. Canary, Atlanta, GA (US); Christopher K. Brooks, Lawrenceville, GA (US); Nathaniel Barnes, Covington, GA (US)

(73) Assignee: C. R. Bard, Inc., Franklin Lakes, NJ (US)

(21) Appl. No.: 18/272,332

(22) PCT Filed: Jan. 11, 2022
(86) PCT No.: PCT/US2022/011971

§ 371 Date: Jul. 13, 2023

Related U.S. Application Data
(60) Provisional application No. 63/138,242, filed on Jan. 15, 2021.

(51) Int. Cl. A61F 7/10; A61F 7/00

(52) U.S. Cl. CPC A61F 7/10; A61F 7/0085; A61F 2007/0226

(57) ABSTRACT

Disclosed herein are systems, pads, and methods thereof for targeted temperature management. A system, for example, can include a control module and a pad. The pad can include a multilayered pad body and one or more comfort-enhancing features integrated into the pad for mitigating or eliminating skin irritation along at least edges of the pad. The multilayered pad body can include a conduit layer, an impermeable film over the conduit layer, and an adherable layer over the impermeable film. The conduit layer can include one or more conduits configured to convey a fluid through the conduit layer. The impermeable film can be configured to retain the fluid in the conduit layer when the fluid is conveyed through the conduit layer. The adherable layer can be configured to adhere to a body portion of a patient. Methods of the systems and pads can include methods of use.

### PRIORITY

This application claims the benefit of priority to U.S. Provisional Patent Application No. 63/138,242, filed Jan. 15, 2021, which is incorporated by reference in its entirety into this application.

### BACKGROUND

Current systems for target temperature management (“TTM”) require adherable pads adhered to patients in order to maintain sufficient contact between the pads and the patients for inducing therapeutic hypothermia by circulating chilled fluid through the pads. However, such pads can cause skin irritation along edges of the pads. What is needed are pads that mitigate or eliminate the foregoing skin irritation along at least the edges of the pads, thereby enhancing comfort for the patients during TTM.

Disclosed herein are TTM systems, pads, and methods thereof that address the foregoing.

### SUMMARY

Disclosed herein is a pad for TTM including, in some embodiments, a multilayered pad body and one or more comfort-enhancing features integrated into the pad for mitigating or eliminating skin irritation along at least edges of the pad. The multilayered pad body includes a conduit layer, an impermeable film over the conduit layer, and an adherable layer over the impermeable film. The conduit layer includes one or more conduits configured to convey a fluid through the conduit layer. The impermeable film is configured to retain the fluid in the conduit layer when the fluid is conveyed through the conduit layer. The adherable layer is configured to adhere to a body portion of a patient.

In some embodiments, the edges of the pad are chamfered for enhanced comfort.

In some embodiments, the edges of the pad are ensconced in a bumper for enhanced comfort.

In some embodiments, the bumper is a tube.

In some embodiments, the conduit layer includes a perimetrical wall and one or more inner walls extending from the conduit layer toward the impermeable film. The perimetrical wall and the one-or-more inner walls form the one-or-more conduits of the conduit layer.

In some embodiments, the conduit layer includes a plurality of protrusions extending from the conduit layer toward the impermeable film. The protrusions are configured to promote even flow of the fluid when the fluid is conveyed through the conduit layer.

In some embodiments, the conduit layer includes a plurality of dimples extending into the conduit layer toward the impermeable film. The dimples are configured to increase a surface area of an exterior surface of the pad and enhance cooling of the pad for enhanced comfort.

In some embodiments, the dimples extending into the conduit layer and the protrusions extending from the conduit layer are coincident.

In some embodiments, each layer of the multilayered pad body is transparent. Transparency allows visual inspection for skin irritation under the pad and along the edges of the pad.

In some embodiments, the pad further includes one or more inlets configured for charging the conduit layer with the fluid and one or more outlets configured for discharging the fluid from the conduit layer.

Also disclosed is a system for TTM including, in some embodiments, a control module and pad. The control module includes a hydraulic system. The hydraulic system includes a chiller evaporator, one or more outlets, and one or more inlets. The chiller evaporator is configured for chilling a fluid to produce a chilled fluid. The one-or-more outlets are configured for discharging the chilled fluid from the hydraulic system. The one-or-more inlets are configured for charging the hydraulic system with the fluid to continue to produce the chilled fluid. The pad includes a multilayered pad body and one or more comfort-enhancing features integrated into the pad for mitigating or eliminating skin irritation along at least edges of the pad. The multilayered pad body includes a conduit layer, an impermeable film over the conduit layer, and an adherable layer over the impermeable film. The conduit layer includes one or more conduits configured to convey the chilled fluid through the conduit layer. The impermeable film is configured to retain the chilled fluid in the conduit layer. The adherable layer is configured to adhere to a body portion of a patient.

In some embodiments, the edges of the pad are chamfered for enhanced comfort.

In some embodiments, the edges of the pad are ensconced in a bumper for enhanced comfort.

In some embodiments, the bumper is a tube.

In some embodiments, the conduit layer includes a perimetrical wall and one or more inner walls extending from the conduit layer toward the impermeable film. The perimetrical wall and the one-or-more inner walls form the one-or-more conduits of the conduit layer.

In some embodiments, the conduit layer includes a plurality of protrusions extending from the conduit layer toward the impermeable film. The protrusions are configured to promote even flow of the chilled fluid when the fluid is conveyed through the conduit layer.

In some embodiments, the conduit layer includes a plurality of dimples extending into the conduit layer toward the impermeable film. The dimples are configured to increase a surface area of an exterior surface of the pad and enhance cooling of the pad for enhanced comfort.

In some embodiments, the dimples extending into the conduit layer and the protrusions extending from the conduit layer are coincident.

In some embodiments, each layer of the multilayered pad body is transparent. Transparency allows visual inspection for skin irritation under the pad and along the edges of the pad.

In some embodiments, the pad further includes one or more inlets configured for charging the conduit layer with the chilled fluid and one or more outlets configured for discharging the chilled fluid from the conduit layer.

Also disclosed herein is a method of a system for TTM. The method includes, in some embodiments, a connecting step, a pad-adhering step, and a hypothermia-inducing step. The connecting step includes connecting an inlet and an outlet of a pad to a hydraulic system of a control module. The pad includes a multilayered pad body having a conduit layer configured to convey a fluid. The pad-adhering step includes adhering the pad to a body portion of a patient by way of an adherable layer of pad. The hypothermia-inducing step includes inducing therapeutic hypothermia in the patient by circulating a chilled fluid provided by the control module through the conduit layer while enhancing comfort of the patient by mitigating of eliminating skin irritation along at least edges of the pad.

In some embodiments, the edges of the pad are chamfered, thereby enhancing the comfort of the patient.

In some embodiments, the edges of the pad are ensconced in a bumper, thereby enhancing the comfort of the patient.

In some embodiments, an exterior surface of the pad is dimpled to increase a surface area of the exterior surface of the pad and enhance cooling, thereby enhancing the comfort of the patient.

In some embodiments, the method further includes an inspecting step. The inspecting step includes visually inspecting for skin irritation under the pad or along the edges of the pad. Each layer of the multilayered pad body is transparent for the inspecting step.

In some embodiments, the connecting step further includes connecting the inlet of the pad to an outlet of the hydraulic system in parallel with one or more other pads of the system. The connecting step also includes connecting the outlet of the pad to an inlet of the hydraulic system in parallel with the one-or-more other pads of the system.

These and other features of the concepts provided herein will become more apparent to those of skill in the art in view of the accompanying drawings and following description, which describe particular embodiments of such concepts in greater detail.

### DRAWINGS

FIG. 1 illustrates a TTM system in accordance with some embodiments.

FIG. 2A illustrates TTM pads for a torso of a patient in accordance with some embodiments.

FIG. 2B illustrates TTM pads for legs of a patient in accordance with some embodiments.

FIG. 3A illustrates a first comfort-enhancing feature of a TTM pad in accordance with some embodiments.

FIG. 3B illustrates a second comfort-enhancing feature of a TTM pad in accordance with some embodiments.

FIG. 3C illustrates a third comfort-enhancing feature of a TTM pad in accordance with some embodiments.

FIG. 4 illustrates a multilayered pad body of a TTM pad in accordance with some embodiments.

FIG. 5 illustrates a hydraulic system of a control module in accordance with some embodiments.

### DESCRIPTION

Before some particular embodiments are disclosed in greater detail, it should be understood that the particular embodiments disclosed herein do not limit the scope of the concepts provided herein. It should also be understood that a particular embodiment disclosed herein can have features that can be readily separated from the particular embodiment and optionally combined with or substituted for features of any of a number of other embodiments disclosed herein.

Regarding terms used herein, it should also be understood the terms are for the purpose of describing some particular embodiments, and the terms do not limit the scope of the concepts provided herein. Ordinal numbers (e.g., first, second, third, etc.) are generally used to distinguish or identify different features or steps in a group of features or steps, and do not supply a serial or numerical limitation. For example, “first,” “second,” and “third” features or steps need not necessarily appear in that order, and the particular embodiments including such features or steps need not necessarily be limited to the three features or steps. Labels such as “left,” “right,” “top,” “bottom,” “front,” “back,” and the like are used for convenience and are not intended to imply, for example, any particular fixed location, orientation, or direction. Instead, such labels are used to reflect, for example, relative location, orientation, or directions. Singular forms of “a,” “an,” and “the” include plural references unless the context clearly dictates otherwise.

Unless defined otherwise, all technical and scientific terms used herein have the same meaning as commonly understood by those of ordinary skill in the art.

As set forth above, current systems for TTM require adherable pads adhered to patients in order to maintain sufficient contact between the pads and the patients for inducing therapeutic hypothermia by circulating chilled fluid through the pads. However, such pads can cause skin irritation along edges of the pads. What is needed are pads that mitigate or eliminate the foregoing skin irritation along at least the edges of the pads, thereby enhancing comfort for the patients during TTM.

Disclosed herein are TTM systems, pads, and methods thereof that address the foregoing.

### TTM Systems

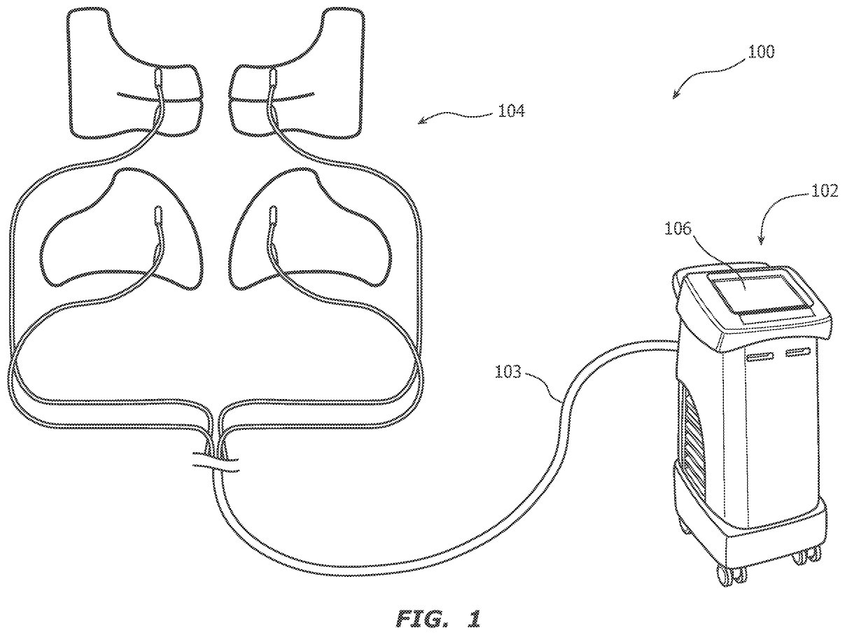

FIG. 1 illustrates a TTM system 100 in accordance with some embodiments.

As shown, the system 100 can include a control module 102, one or more TTM pads 104 such as those set forth below, and one or more fluid conduits 103 therebetween. Description for the control module 102 is set forth immediately below. Description for the one-or-more pads 104 is set forth in a following section.

The control module 102 can include a console 106 with an integrated display screen configured as a touchscreen for operating the control module 102. The console 106 can include one or more processors, primary and secondary memory, and instructions stored in the primary memory configured to instantiate one or more processes for TTM with the control module 102.

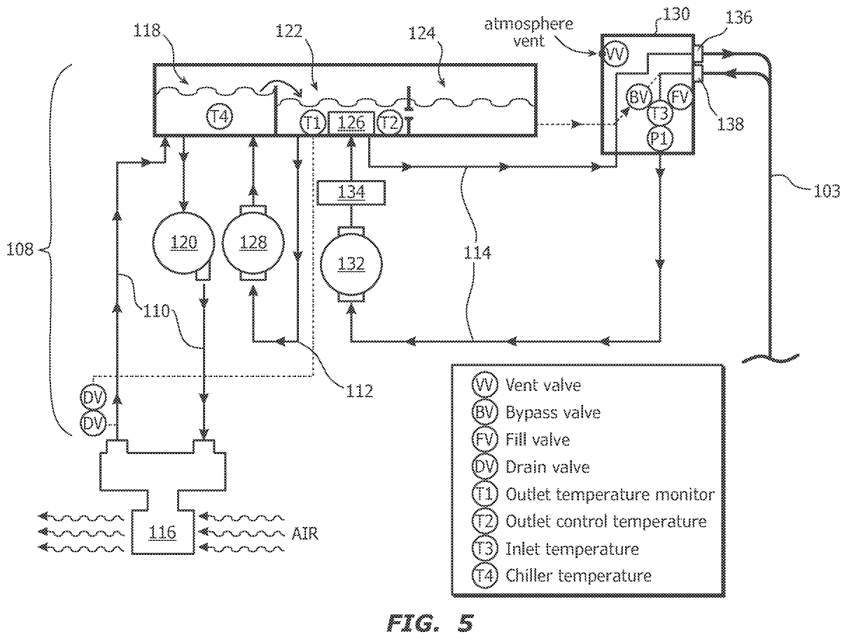

FIG. 5 illustrates a hydraulic system 108 of the control module 102 in accordance with some embodiments.

The control module 102 can also include the hydraulic system 108, which can include a chiller circuit 110, a mixing circuit 112, and a circulating circuit 114.

The chiller circuit 110 can be configured for chilling a fluid (e.g., water, ethylene glycol, a combination of water and ethylene glycol, etc.) to produce a chilled fluid, which chilled fluid, in turn, can be for mixing with an optionally heated fluid to produce a supply fluid for TTM. The chiller circuit 110 can include a chiller evaporator 116 configured for the chilling of the fluid passing therethrough. The fluid for the chilling by the chiller evaporator 116 is provided by a chiller tank 118 using a chiller pump 120 of the chiller circuit 110.

The mixing circuit 112 can be configured for mixing spillover of the chilled fluid from the chiller tank 118 with a mixed fluid in a mixing tank 122 of the mixing circuit 112. The mixing circuit 112 can include a heater 126 in the mixing tank 122 configured for heating the mixed fluid to produce a heated fluid if needed for mixing with the chilled fluid to provide a supply tank 124 of the circulating circuit 114 with a supply fluid of a desired temperature for TTM. The mixing circuit 112 can include a mixing pump 128 configured to pump the fluid from the mixing tank 122 into the chiller tank 118 for producing the chilled fluid and the spillover of the chilled fluid for the mixing tank 122.

The circulating circuit 114 can be configured for circulating the supply fluid for TTM, which includes circulating the supply fluid provided by the manifold 130 through the one-or-more pads 104 using a circulation pump 132 directly or indirectly governed by a flow meter 134 of the circulating circuit 114. The manifold 130 can include one or more outlets 136 configured for discharging the supply fluid (e.g., a chilled fluid) from the hydraulic system 108 and one or more inlets 138 configured for charging the hydraulic system 108 with return fluid from the one-or-more pads 104 to continue to produce the supply fluid.

### TTM Pads

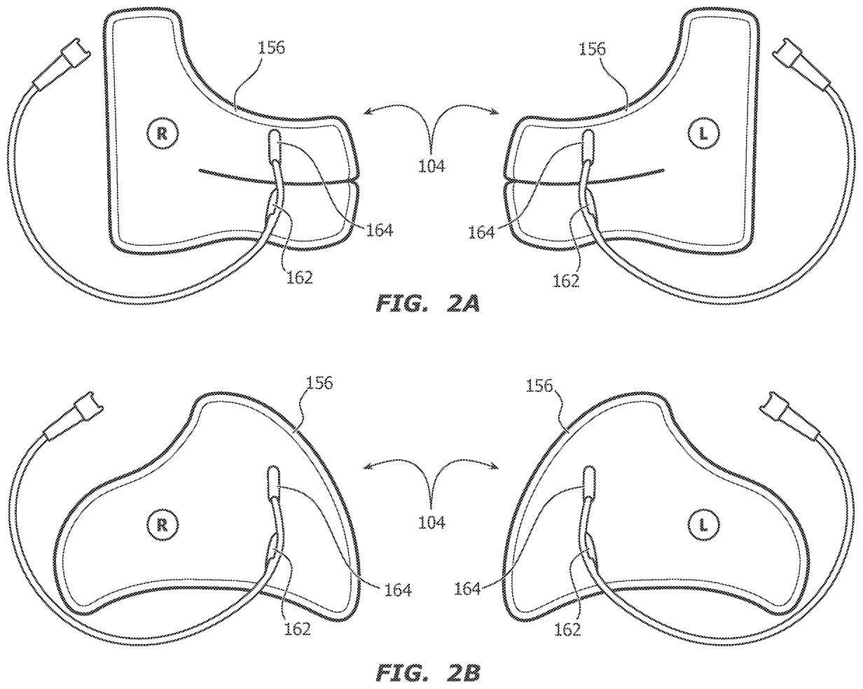

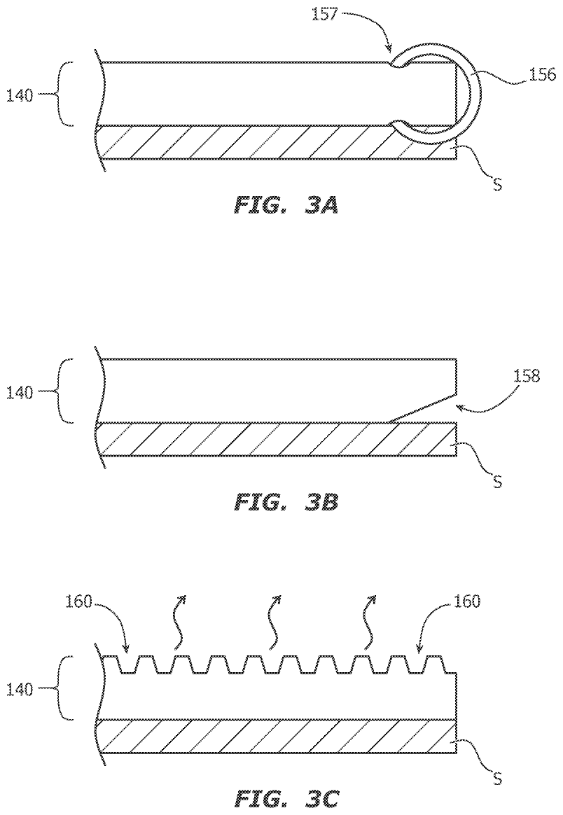

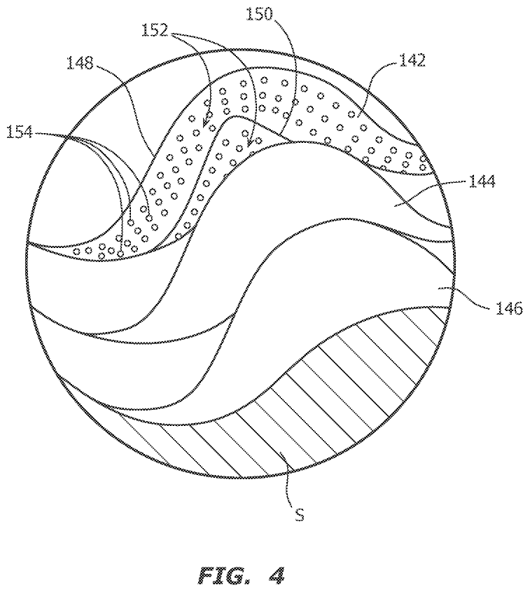

FIGS. 2A and 2B illustrate left and right pads of the one-or-more pads 104 having comfort-enhancing features respectively for a torso and legs of a patient in accordance with some embodiments. FIGS. 3A-3C illustrate different comfort-enhancing features of the one-or-more pads 104 in accordance with some embodiments. FIG. 4 illustrates a multilayered pad body 140 of a pad of the-one-or-more pads 104 in accordance with some embodiments.

A pad of the one-or-more pads 104 can include the multilayered pad body 140 and one or more comfort-enhancing features integrated into the pad for mitigating or eliminating skin irritation.

The multilayered pad body 140 can include a conduit layer 142, an impermeable film 144 over the conduit layer 142, and an adherable layer 146 over the impermeable film 144.

The conduit layer 142 includes a perimetrical wall 148 and one or more inner walls 150 extending from the conduit layer 142 toward the impermeable film 144. Together, the perimetrical wall 148 and the one-or-more inner walls 150 form one or more conduits 152 configured to convey a fluid through the conduit layer 142.

The conduit layer 142 can include a plurality of protrusions 154 extending from the conduit layer 142 toward the impermeable film 144. The protrusions 154 are configured to promote even flow of the fluid when the fluid is conveyed through the conduit layer 142.

The conduit layer 142 can be of an insulating foam. Depending upon a desired effect, the insulating foam can be configured to prevent or encourage heat loss into an ambient environment.

The impermeable film 144 can be configured to retain the fluid in the conduit layer 142 when the fluid is conveyed through the conduit layer 142. In addition, the impermeable film 144 can be configured to allow efficient energy transfer from the fluid in the conduit layer 142 through the adherable layer 146.

The adherable layer 146 can be configured to adhere to a body portion (e.g., torso, leg, etc.) of a patient for direct thermal conduction through skin S of the patient.

The adherable layer 146 can include a biocompatible hydrogel in a 50% water-polymer matrix to provide uniform skin contact for the direct thermal conduction.

The one-or-more comfort-enhancing features integrated into a pad of the one-or-more pads 104 for mitigating or eliminating skin irritation can include comfort-enhancing features along at least edges of the pad, which edges can be otherwise sharp and cause the skin irritation.

As shown in FIGS. 2A, 2B, and 3A, the edges of the pad can be ensconced in a bumper 156 for enhanced comfort. The bumper 156 can be a soft, flexible tube split along its length a coupled to the pad by way of insertion into a groove 157 around the pad. The tube can be of a foam or an elastomeric material configured to become more pliable when heated for insertion of the tube into the groove 157 around the pad.

As shown in FIG. 3B, the edges of the pad can be chamfered with a chamfer 158 for enhanced comfort.

The one-or-more comfort-enhancing features integrated into a pad of the one-or-more pads 104 for mitigating or eliminating skin irritation can include comfort-enhancing features over an entirety of the pad.

As shown in FIG. 3C, the conduit layer 142 can include a plurality of dimples 160 extending into the conduit layer 142 toward the impermeable film 144. The dimples 160 can be configured to increase a surface area of an exterior surface of the pad and enhance cooling of the pad for enhanced comfort. Indeed, in this case, the insulating foam of the conduit layer 142 can be configured to encourage heat loss into an ambient environment. Advantageously, the dimples 160 extending into the conduit layer 142 and the protrusions 154 extending from the conduit layer 142 are coincident. That is, a dimple of the dimples 160 extending into the conduit layer 142 from the exterior surface can, on an opposite side of the conduit layer, be a protrusion of the protrusions 154 extending from the conduit layer 142 toward the impermeable film 144.

For convenience, each layer of the multilayered pad body 140 can be transparent. Transparency allows visual inspection for skin irritation under the pad and along the edges of the pad.

In addition to the foregoing, the pad can include one or more inlets 162 and one or more outlets 164. The one-or-more inlets 162 are configured for charging the conduit layer 142 with the fluid, while the one-or-more outlets 164 are configured for discharging the fluid from the conduit layer 142.

### Methods

Methods of the systems and pads include methods of use. For example, a method of using the system 100 includes a connecting step, a pad-adhering step, and a hypothermia-inducing step.

The connecting step includes connecting an inlet of the one-or-more inlets 162 and an outlet of the one-or-more outlets of a pad of the one-or-more pads 104 to the hydraulic system 108 of the control module 102. The connecting step can further include connecting the inlet of the pad to an outlet of the one-or-more outlets 136 of the hydraulic system 108 in parallel with one or more other pads of the system 100. The connecting step can further include connecting the outlet of the pad to an inlet of the one-or-more inlets 138 of the hydraulic system 108 in parallel with the one-or-more other pads of the system 100.

The pad-adhering step includes adhering the pad to a body portion (e.g., torso, leg, etc.) of a patient by way of the adherable layer 146 of pad.

The hypothermia-inducing step includes inducing therapeutic hypothermia in the patient by circulating a chilled fluid provided by the control module 102 through the conduit layer 142 while enhancing comfort of the patient by mitigating of eliminating skin irritation along at least edges of the pad. As set forth above, the edges of the pad can be ensconced in the bumper 156, thereby enhancing the comfort of the patient during the hypothermia-inducing step. Alternatively, the edges of the pad can be chamfered, thereby enhancing the comfort of the patient during the hypothermia-inducing step. Further as set forth above, the exterior surface of the pad can be dimpled to increase the surface area of the exterior surface of the pad and enhance cooling, thereby enhancing the comfort of the patient during the hypothermia-inducing step.

The method can further include an inspecting step. The inspecting step can include visually inspecting for skin irritation under the pad or along the edges of the pad. A set forth above, each layer of the multilayered pad body 140 can be transparent for the inspecting step.

While some particular embodiments have been disclosed herein, and while the particular embodiments have been disclosed in some detail, it is not the intention for the particular embodiments to limit the scope of the concepts provided herein. Additional adaptations and/or modifications can appear to those of ordinary skill in the art, and, in broader aspects, these adaptations and/or modifications are encompassed as well. Accordingly, departures may be made from the particular embodiments disclosed herein without departing from the scope of the concepts provided herein.

1. A pad for targeted temperature management (“TTM”), comprising:
a multilayered pad body including:
a conduit layer including one or more conduits configured to convey a fluid through the conduit layer;
an impermeable film over the conduit layer configured to retain the fluid in the conduit layer when the fluid is conveyed through the conduit layer; and
an adherable layer over the impermeable film configured to adhere to a body portion of a patient; and
one or more comfort-enhancing features integrated into the pad for mitigating or eliminating skin irritation along at least edges of the pad.

2. The pad of claim 1, wherein the edges of the pad are chamfered for enhanced comfort.

3. The pad of claim 1, wherein the edges of the pad are ensconced in a bumper for enhanced comfort.

4. The pad of claim 3, wherein the bumper is a tube.

5. The pad of claim 1, wherein the conduit layer includes a perimetrical wall and one or more inner walls extending from the conduit layer toward the impermeable film, the perimetrical wall and the one-or-more inner walls forming the one-or-more conduits of the conduit layer.

6. The pad of claim 1, wherein the conduit layer includes a plurality of protrusions extending from the conduit layer toward the impermeable film, the protrusions configured to promote even flow of the fluid when the fluid is conveyed through the conduit layer.

7. The pad of claim 6, wherein the conduit layer includes a plurality of dimples extending into the conduit layer toward the impermeable film, the dimples configured to increase a surface area of an exterior surface of the pad and enhance cooling of the pad for enhanced comfort.

8. The pad of claim 7, wherein the dimples extending into the conduit layer and the protrusions extending from the conduit layer are coincident.

9. The pad of claim 1, wherein each layer of the multilayered pad body is transparent, thereby allowing visual inspection for skin irritation under the pad and along the edges of the pad.

10. The pad of claim 1, further comprising one or more inlets configured for charging the conduit layer with the fluid and one or more outlets configured for discharging the fluid from the conduit layer.

11. A system for targeted temperature management (“TTM”), comprising:
a control module including a hydraulic system having:
a chiller evaporator configured for chilling a fluid to produce a chilled fluid;
one or more outlets configured for discharging the chilled fluid from the hydraulic system; and
one or more inlets configured for charging the hydraulic system with the fluid to continue to produce the chilled fluid; and
a pad including:
a multilayered pad body having:
a conduit layer including one or more conduits configured to convey the chilled fluid through the conduit layer;
an impermeable film over the conduit layer configured to retain the chilled fluid in the conduit layer; and
an adherable layer over the impermeable film configured to adhere to a body portion of a patient; and
one or more comfort-enhancing features integrated into the pad for mitigating or eliminating skin irritation along at least edges of the pad.

12. The system of claim 11, wherein the edges of the pad are chamfered for enhanced comfort.

13. The system of claim 11, wherein the edges of the pad are ensconced in a bumper for enhanced comfort.

14. The system of claim 13, wherein the bumper is a tube.

15. The system of claim 1, wherein the conduit layer includes a perimetrical wall and one or more inner walls extending from the conduit layer toward the impermeable film, the perimetrical wall and the one-or-more inner walls forming the one-or-more conduits of the conduit layer.

16. The system of claim 1, wherein the conduit layer includes a plurality of protrusions extending from the conduit layer toward the impermeable film, the protrusions configured to promote even flow of the chilled fluid.

17. The system of claim 16, wherein the conduit layer includes a plurality of dimples extending into the conduit layer toward the impermeable film, the dimples configured to increase a surface area of an exterior surface of the pad and enhance cooling of the pad for enhanced comfort.

18. The system of claim 17, wherein the dimples extending into the conduit layer and the protrusions extending from the conduit layer are coincident.

19. The system of claim 11, wherein each layer of the multilayered pad body is transparent, thereby allowing visual inspection for skin irritation under the pad and along the edges of the pad.

20. The system of claim 11, further comprising one or more inlets configured for charging the conduit layer with the chilled fluid and one or more outlets configured for discharging the chilled fluid from the conduit layer.

21. A method for targeted temperature management (“TTM”), comprising:
connecting an inlet and an outlet of a pad to a hydraulic system of a control module, the pad including a multilayered pad body having a conduit layer configured to convey a fluid;
adhering the pad to a body portion of a patient by way of an adherable layer of pad; and
inducing therapeutic hypothermia in the patient by circulating a chilled fluid provided by the control module through the conduit layer while enhancing comfort of the patient by mitigating of eliminating skin irritation along at least edges of the pad.

22. The method of claim 21, wherein the edges of the pad are chamfered, thereby enhancing the comfort of the patient.

23. The method of claim 21, wherein the edges of the pad are ensconced in a bumper, thereby enhancing the comfort of the patient.

24. The method of claim 21, wherein an exterior surface of the pad is dimpled to increase a surface area of the exterior surface of the pad and enhance cooling, thereby enhancing the comfort of the patient.

25. The method of claim 21, further comprising visually inspecting for skin irritation under the pad or along the edges of the pad, each layer of the multilayered pad body being transparent for the inspecting.

26. The method of claim 21, wherein connecting the inlet of the pad to the hydraulic system of the control module includes connecting the inlet to an outlet of the hydraulic system in parallel with one or more other pads of the system and connecting the outlet of the pad to the hydraulic system of the control module includes connecting the outlet to an inlet of the hydraulic system in parallel with the one-or-more other pads of the system.
